(19) United States
(12) Patent Application Publication
(10) Pub. No.: US 2022/0003770 A1
(43) Pub. Date: Jan. 6, 2022

(54) Classification and Treatment of Gastric Cancer

(71) Applicant: The General Hospital Corporation, Boston, MA (US)

(72) Inventors: Gregory Y. Lauwers, Tierra Verde, FL (US); Gabriel Dan Duda, Belmont, MA (US)

(21) Appl. No.: 17/371,954

(22) Filed: Jul. 9, 2021

Related U.S. Application Data
(63) Continuation of application No. 16/076,906, filed on Aug. 9, 2018, now abandoned.
(60) Provisional application No. 62/293,063, filed on Feb. 9, 2016.

(51) Int. Cl. G01N 33/574; G01N 33/569

(52) U.S. Cl. CPC G01N 33/57446; G01N 33/56994; G01N 2333/05; G01N 2333/70596; G01N 2333/4748; G01N 2333/9126; G01N 2333/4725

(57) ABSTRACT

Protein and mRNA expression based methods for classification of gastric cancer, and methods of treatment based thereon.

### CLAIM OF PRIORITY

This application is a continuation of U.S. patent application Ser. No. 16/076,906, filed Aug. 9, 2018, which is a § 371 national stage application of International Application No. PCT/US2017/017195, filed on Feb. 9, 2017, which claims the benefit of U.S. Provisional Application Ser. No. 62/293,063, filed on Feb. 9, 2016. The entire contents of the foregoing are incorporated herein by reference.

### TECHNICAL FIELD

Described herein are protein and mRNA expression based methods for classification of gastric cancer, and methods of treatment based thereon.

### BACKGROUND

Gastric carcinoma (GC) is the fifth most common malignancy and the third leading cause of cancer-related death worldwide. Despite better control over known risk factors and development of new chemotherapeutic and targeted agents, in the United States, the 5-year overall survival for GC patients is only 29%.¹ In regard to patient stratification, although several morphologic classifications have been developed, one broadly used system is the Lauren classification, which divides GC into intestinal and diffuse types.² The simple two-tiered classification gives a general understanding of the histogenesis and biology of GC, and has been particularly helpful in evaluating epidemiologic data. Another widely used system is the WHO classification, which is based on more precise histologic patterns. It is an all-inclusive system, which recognizes all the rare subtypes that were not identified in the Lauren classification.³ Nevertheless, its clinical utility is doubtful since there is little difference in outcomes between the distinct histological subgroups.

### SUMMARY

The overall survival of gastric carcinoma (GC) patients remains poor despite improved control over known risk factors and surveillance. This highlights the need for new classifications, driven towards identification of potential therapeutic targets. Using sophisticated molecular technologies and analysis, three groups recently provided genetic and epigenetic molecular classifications of gastric cancer (The Cancer Genome Atlas (TCGA), “Singapore-Duke” study, and Asian Cancer Research Group (ACRG)). Herein, the expression of fourteen biomarkers was examined in a cohort of 146 GCs and unsupervised hierarchical clustering analysis was performed using less expensive and widely available immunohistochemistry and in situ hybridization. Ultimately, five groups of GCs were identified based on Epstein Barr virus (EBV)-positivity, microsatellite Instability (MSI), aberrant E-cadherin and p53 expression; the remaining cases constituted a group characterized by normal p53 expression. In addition, the five categories correspond to the reported molecular subgroups by virtue of clinicopathologic features. Furthermore, evaluation between these clusters and survival using the Cox proportional hazards model showed a trend for superior survival in the EBV and MSI-related GCs. Provided herein is a simplified algorithm that is able to identify five subgroups (also referred to herein as clusters) of GC, using immunohistochemical and in situ hybridization techniques, that can be used, e.g., to stratify patients and to select optimal treatments.

Thus, provided herein are methods comprising providing a sample comprising tissue comprising tumor cells from a gastric tumor; and determining in tumor cells in the sample:

(i) a level of Epstein Barr Virus in the tumor cells;

(ii) DNA mismatch repair protein expression in the tumor cells;

(iii) E-cadherin protein expression in the tumor cells;

(iv) p53 protein expression in the tumor cells; and

(v) Mucin protein expression in the tumor cells.

Also provided herein are methods for categorizing gastric tumor in a subject. The methods can include providing a sample comprising tissue comprising tumor cells from a gastric tumor; and determining in tumor cells in the sample:

(i) a level of Epstein Barr Virus (EBV) in the tumor cells, preferably a nuclear level of EBV;

(ii) DNA mismatch repair protein expression in the tumor cells;

(iii) E-cadherin protein expression in the tumor cells;

(iv) p53 protein expression in the tumor cells; and

(v) Mucin protein expression in the tumor cells; and

categorizing a tumor with EBV, preferably nuclear EBV, above a reference level, and optionally with prominent lymphoid infiltrate, in group 1;
categorizing a tumor with a nuclear DNA mismatch repair protein expression below a reference level in group 2;
categorizing a tumor with E-cadherin expression below a reference level, or only cytoplasmic E-cadherin expression, in group 3;
categorizing a tumor with normal p53 expression and cytoplasmic mucin expression above a reference level in group 4; and
categorizing a tumor with aberrant levels of p53 expression and normal levels of mucin expression in group 5.

In the methods described herein, “determining expression” can include determining presence, level, and/or localization of the protein or virus.

In some embodiments, the level of Epstein Barr Virus is determined using EBER in situ hybridization (EBER-ISH), and/or the DNA mismatch repair protein expression; E-cadherin expression; p53 expression; and Mucin expression is detected using immunohistochemistry in intact tumor samples.

In some embodiments, the mismatch repair proteins comprise one, two, three, or all four of MLH1, PMS2, MSH2, or MSH6.

In some embodiments, the mismatch repair proteins comprise MLH1 and PMS2.

In some embodiments, the Mucin is one or more of MUC2, CDX2, CD10, MUC5AC, or MUC6. In some embodiments, the Mucin is MUC6.

In some embodiments, aberrant p53 expression is an absence of expression or strong diffuse expression, and normal p53 expression is weak, patchy expression.

In some embodiments, the methods include selecting, and optionally administering,

a treatment comprising a therapeutically effective amount of an immunotherapy, e.g.,
a checkpoint inhibitor to a subject having a tumor in group 1;
a treatment comprising a therapeutically effective amount of an immunotherapy, topoisomerase-I inhibitors, or platinum-based chemotherapy, e.g., in combination with poly (ADP-ribose) polymerase (PARP) inhibitors, to a subject having a tumor in group 2;
a treatment comprising a therapeutically effective amount of an inhibitor of mammalian target of rapamycin (mTOR) to a subject having a tumor in group 3;
a treatment comprising a therapeutically effective amount of an antimetabolite to a subject having a tumor in group 4; or
a treatment comprising a therapeutically effective amount of an inhibitor of polo-like kinase 1 (PLK1) and/or an inhibitor of Aurora Kinase A to a subject having a tumor in group 5.

Therapeutic agents that can be used in the methods include those known in the art and/or described herein.

In some embodiments, the checkpoint inhibitor is an anti-PD1 or anti-PDL1 antibody, e.g., as known in the art and/or described herein.

In some embodiments, the mTOR inhibitor is rapamycin, ridaforolimus, temsirolimus, everolimus, sirolimus, dactolisib, AZD8055, CZ415, CC-223, voxtalisib, PI-103, KU-0063794, torkinib, ridaforolimus, Voxtalisib (SAR245409, XL765) analogue, omipalisib, OSI-027, PF-04691502, Apitolisib (GDC-0980, RG7422), GSK1059615, Gedatolisib (PF-05212384, PKI-587), WYE-354, Vistusertib (AZD2014), Torin 2, WYE-125132 (WYE-132), BGT226 (NVP-BGT226), Palomid 529 (P529), PP121, WYE-687, CH5132799, WAY-600. ETP-46464. GDC-0349, XL388, zotarolimus, MHY1485 and Chrysophanic Acid, preferably rapamycin, ridaforolimus, temsirolimus, everolimus or sirolimus, e.g., as known in the art and/or described herein.

In some embodiments, the antimetabolite is a pyrimidine analog, preferably 5-Fluorouracil (5-FU), gemcitabine, fluorouracil, or cytarabine, e.g., as known in the art and/or described herein.

In some embodiments, the inhibitor of polo-like kinase 1 (PLK1) is volasertib, BI 2536, GSK461364, NMS-P937 (NMS1286937), Ro3280, MLN0905, SBE 13 HCl, HMN-214, HMN-176, or rigosertib, e.g., as known in the art and/or described herein. In some embodiments, the inhibitor of inhibitor of Aurora Kinase A is MK-5108 (VX-689), MK-8745, Alisertib (MLN8237), Aurora A Inhibitor I, MLN8054, ENMD-2076 L-(+)-Tartaric acid, ENMD-2076, KW-2449, VX-680 (Tozasertib, MK-0457), PF-03814735, AT9283, AMG-900, CYC116, SNS-314 Mesylate, JNJ-7706621, Reversine, Danusertib (PHA-739358), CCT137690, TAK-901, PHA-680632, CCT129202, ZM 447439, GSK1070916, Barasertib (AZD1152-HQPA), alisertib (MLN8327), or Hesperadin, e.g., as known in the art and/or described herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1: Heat map depicting clinicopathologic features and biomarker results for the cohort. Cluster analysis of 146 cases which are represented in the columns and the clinicopathologic features and biomarkers (i.e., the variables studied) in the rows. The variables are shown on the right. Aberrantly expressed variables in the cohort are shown in different shades depending on the fold difference. Variables shown in white were not available for interpretation.

FIGS. 2A-C: EBV-associated Gastric Carcinoma. (A) The neoplasm displays a prominent lymphoid infiltrate. (B) EBV encoded RNA in-situ hybridization showing specific (brown) transcripts in the neoplastic nuclei. (C) EBV-associated Gastric Carcinoma showing strong membranous expression of PD-L1 (immune-checkpoint pathway protein, Programmed Death Ligand 1).

FIG. 3: Kaplan-Meier curves for gastric cancer survival according to protein expression based classification.

FIGS. 4A-C: Microsatellite Instable Gastric Carcinoma showing intestinal phenotype (A), with aberrant overexpression of p53 (B) and loss of MLH1 protein (C). PMS2 was also lost in the tumor cells; however, MSH2 and MSH6 proteins were retained.

FIGS. 5A-D: Aberrant E-cadherin associated Gastric Carcinoma. (A & B) Complete loss of e-cadherin protein is noted in this diffuse type/poorly cohesive adenocarcinoma; (C&D) These pictures depict granular/dot-like expression of e-cadherin protein in a poorly cohesive adenocarcinoma.

FIG. 6: Interpretation of sub-characterization of Cluster 4 based on immunophenotype. Not included is “null phenotype” which is MUC2-, CDX2-, CD10-, MUCSAC-, MUC6-.

FIGS. 7A-I: Gastric Carcinoma of various phenotype: Gastric phenotype, Cluster 4 (A, H&E) showing overexpression of p53 protein (B) and cytoplasmic expression of MUC6 protein (C). Intestinal phenotype, Cluster 4 (D, H&E) showing cytoplasmic expression of MUC2 protein (E) and overexpression of p53 protein (F). Another example with intestinal phenotype, Cluster 5 (G, H&E) with cytoplasmic expression of MUC6 protein (H) but normal pattern of p53 protein (I).

FIG. 8. Summary of protein expression-based classification. The hierarchical clustering resulted in the determination of five groups of gastric adenocarcinomas. EBV, Epstein-Barr virus; GC, gastric cancer; MSI-H, high microsatellite instability.

FIG. 9. Current Molecular Classifications of Gastric Cancer proposed by the “Singapore-Duke” group, the Cancer Genome Atlas (TCGA) and the Asian Cancer Research Group (ACRG) Abbreviations: CIN, chromosomal instability; EBV, pstein-Barr virus; EMT, epithelial-mesenchymal transition; FU, fluorouracil; GS, genomically stable; MSI, microsatellite instability; MSS, microsatellite stable. Proposed by the ‘Singapore-Duke’ group, the Cancer Genome Atlas, and the Asian Cancer Research Group.

### DETAILED DESCRIPTION

Following the successful Trastuzumab for Gastric Cancer (ToGA) trial, the only validated predictive biomarker for personalized therapy of GC is limited to human epidermal growth factor receptor (Her2) protein expression.⁴ Recently, anti-vascular endothelial growth factor receptor 2 (VEGFR2) antibody (ramucirumab) has been FDA approved, and anti-epidermal growth factor receptor (EGFR) and mesenchymal-epithelial transition factor (MET or hepatocyte growth factor receptor therapy) are in clinical development.⁴

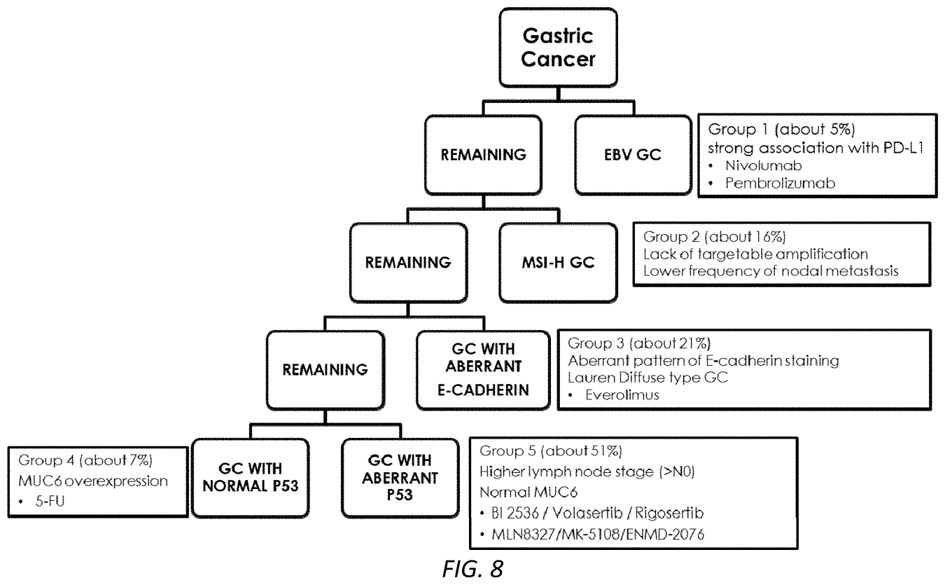

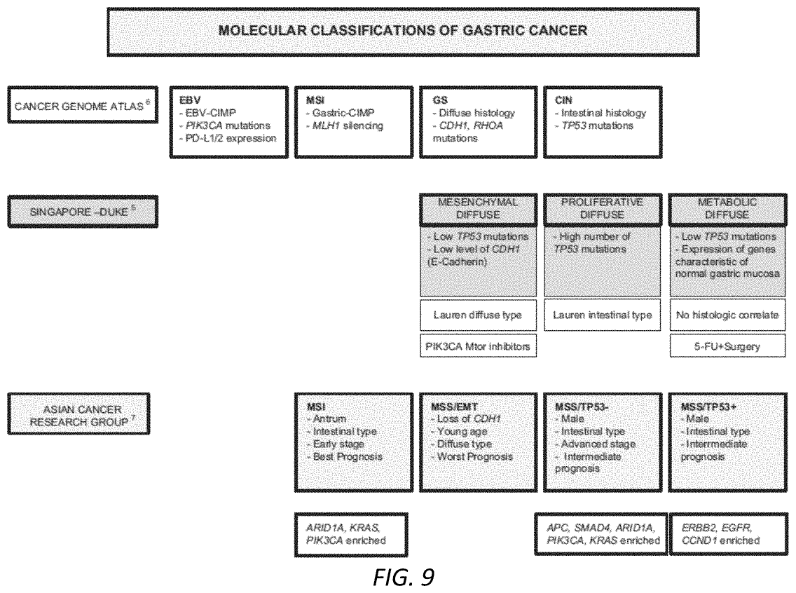

Despite ongoing progress, a certain disconnect persists between the morphologic classification schemes and the biology of GC and, ultimately, the applications of targeted therapies. Recent studies have emphasized the need for new patient stratification strategies that incorporate the emerging molecular classification of GC and identification of potential therapeutic targets (FIG. 8).

The “Singapore-Duke” study identified 3 distinct molecular signatures based on genetic and epigenetic expression of drug responsive clusters, and subclassified GC into (a) a proliferative subtype characterized by high TP53 mutations and activation of oncogenic pathways, (b) a metabolic subtype with low TP53 mutations and expression of genes characteristic of normal gastric mucosa and metabolic pathways, among other features, and (c) a mesenchymal subtype with low expression of CDH1 and TP53, increased stem cell marker, and genes characteristic of the epithelial-mesenchymal transition pathway.⁵ The authors commented that the proliferative and mesenchymal types corresponded to Lauren intestinal and diffuse types, respectively. Metabolic type GCs were highly sensitive to 5-fluorouracil, and the mesenchymal type GCs were more sensitive to phosphatidyl-inositol-3-kinase inhibitors. The Cancer Genome Atlas (TCGA) subdivides GC based on genetic and epigenetic expression in 4 pathogenetic pathways: (a) EBV GCs characterized by hypermethylation of CDKN2A, mutations in PIK3A and PD-L1 expression, (b) MSI-H GCs with hypermethylation and MLH1 silencing, (c) Genomically stable (GS) group with CDH1 and RHOA mutations, and (d) Chromosomal instable (CIN) group corresponding to intestinal histology and a high number of TP53 mutations.⁶ Another recent study from the Asian Cancer Research Group (ACRG) based on gene expression profiling, genome-wide copy number microarrays and targeted gene sequencing proposed classification of GC into (1) MSI GC which present with best overall prognosis and the lowest frequency of recurrence (2) Micro satellite stable (MSS) Epithelial Mesenchymal Transition (EMT) GC with the worst prognosis, (3 & 4) non-MSI and non-EMT TP53 active and inactive GC with intermediate prognosis and recurrence rates.⁶⁵ Although these studies represent a significant progress in defining GC from a biologic point of view, they were limited either by a lack in clinical scope or the lack of immediate clinical applicability. In fact, to define these sub-groups, these studies used multiple advanced molecular techniques including DNA sequencing, RNA sequencing, whole exome sequencing, copy number variation analysis and DNA methylation arrays, which are not currently cost-effective in practice. The present study tested the validity of the three classifications using a series of GCs, focusing on protein and mRNA expression and using techniques available in routine diagnostic practice. In addition, an algorithm was formulated to distinguish the molecular subtypes for easy clinical classification and better patient selection for putative targeted therapy.

Described herein is a biomarker expression-based classification of GCs that parallels the recently recognized genetic classifications of GC.^{5,6, 65} The hierarchical clustering resulted in the determination of five groups of adenocarcinomas.

Group 1: EBV-associated gastric cancers. They represented 5% of the cases. Those were associated with a better survival and a strong association with PD-L1.

Group 2: MSI-H gastric cancers. This group represented 16% overall of the cases. Those were associated with a better survival and a lower frequency of nodal metastases.

Group 3: Gastric cancers with aberrant E-cadherin expression. 21% of the cases fell in this group. Those were associated with diffuse type phenotype.

Group 4: Gastric cancers with aberrant TP53 expression. These represented 51% of the cases. Those were associated with higher lymph node stage and an intestinal phenotype (Lauren classification).

Group 5 grouped gastric cancers with normal TP53 expression and represented 7% of the cases. Those were associated with MUC6 overexpression.

A schema for diagnostic distinction and subsequent treatment is provided in FIG. 8. As these groups may not entirely mutually exclusive, in some embodiments, both treatments can be administered, e.g., one can be tried first or they can be administered simultaneously.

In the cohort, there was a lower incidence of EBV GCs (5%) compared to the TCGA⁶ (8.8%) and other publications (2-20%).⁸ However, another study showed a similar frequency of 5.1%.¹⁸ The mechanism of EBV-carcinogenesis is a genome-wide DNA methylation. The process involves a series of tumor suppressor genes (e.g. p14, p15, p16, APC, CDH, MGMT and PTEN) that result in uncontrolled cell growth.^{22,23}

Of note, although MSI GCs are also associated with hypermethylation and silencing of genes (and their promoters) in a distinctive; pattern. EBV GCs having a more extensive pattern of methylation of promoter and non-promoter CpG islands.²⁴ In fact, EBV GCs are almost exclusive of the microsatellite instable phenotype.^{6,25-27} A series of characteristics important to underscore, EBV GCs lack RHOA mutations;^{6,25,26} however, a decreased expression of RHOA (and CDH1) may be seen secondary to hypermethylation of the gene or its promoter.^{20,25,26,28} They also show a lack of TP53 mutations.^{6,25,26,29,30} This trend was also recorded in ACGR data set.⁶⁵ It has been suggested that the profound global DNA effects are responsible for the distinctive clinico-pathologic features of EBV GCs²² including male predominance^{8,31} and proximal location.³² Notably, the purported younger age has not been confirmed by recent analysis.^{18,33} In the current study, we confirmed the non-antral location. A trend for younger (<70 years) male patients was seen; however, the difference was not statistically significant. A prominent lymphoid infiltrate was seen in all cases, which is consistent with prior studies.³⁴ This group also had trend towards better survival, consistent with the reported literature.^{25,33,35-37}

A significant association with the immune-checkpoint pathway protein programmed death ligand (PD-L1) was seen in the EBV GC cluster. Similar findings have been noted at genomic levels with amplification of the 9p24.1 locus which includes the CD274 gene (encoding for PD-L1).^{6,26} The prognostic significance of this finding is unclear currently. Nevertheless, it suggests a potential role for immunotherapy with anti-PD-1/PD-L1 monoclonal antibodies augmenting antitumor immune response.

MSI GCs have been recognized previously as a distinct group based on clinicopathologic³⁸ and molecular findings.^{6,39} However, aberrant E-cadherin or p53 expression may be seen in this subgroup, with hypermethylation of CDH1²⁸ and abnormalities in TP53 (most frequently as a result of loss of heterozygosity).⁴⁰ There is no prognostic significance to these findings, allowing the present segregation strategy. The frequency of MSI GC in the present cohort was 16% versus 21.7% in the TCGA⁶, 22.7% in the ACRG⁶⁵ and 8%-25.9% in the literature.⁴¹⁻⁴³ The reported incidence is higher in western studies⁴⁴ and associated with older age, female gender, larger tumor size, intestinal differentiation, and lower rate of nodal involvement.^{41,45} In the ACRG, the MSI subtype was predominantly associated with antral location, early stage and intestinal phenotype as well.⁶⁵ The association with intestinal differentiation as established by MUC2 positivity (rather than the Lauren intestinal morphotype) and lower frequency of nodal metastasis was confirmed. This observation is also reflective of lesser biologic aggressiveness and a trend towards longer survival in this group. Consistent with prior studies, loss of MLH1 and PMS2 was the predominant pattern.^{41,46} The mechanism is predominantly hypermethylation of MLH1 promoter^{6,47} and, less commonly, mutations in MLH1 and MSH2.⁴⁷ Notably, MMR deficiency and TP53 mutations are not mutually exclusive carcinogenic pathways. Similar observations have been noted in prior studies in GC.⁴⁰

After exclusion of EBV and MSI clusters, a distinct subgroup of cases characterized by aberrant E-cadherin expression was identified. A few studies have reported TP53 mutations as a late event in the development of poorly cohesive GC, which was our rationale for using cases of “GC with aberrant E-cadherin expression” before “GC with aberrant p53 expression” to assign a group for clinical applicability.^{49,50} The frequency of this subset best correlates with the GC stable group (GS) reported by Bass,⁶ and the MSS/EMT subtype of the ACRG.⁶⁵ This group constituted 21% of the entire cohort similar to the 20% and 15.3% reported in these studies. The histologic Lauren diffuse type, observed in this group (90% diffuse type), is comparable to the so-called mesenchymal group of Lei et al⁵ (seen in 73% diffuse type). An enrichment of mutations in CDH1 has been noted in 37% of the GS group; and an additional 30% of the cases had either RHOA or CLDN18-affecting RHOA or ARHGAP's regulation of RHOA and/or cell motility.⁶ Of note, activated RHOA has been reported to act through effectors, including ROCK1, with resultant activation of STAT3, whose expression has been linked to aberrant E-cadherin expression in diffuse type GCs.^{51,52} Although not confirmed, it is likely that genetic and epigenetic changes in both CDH1 and RHOA would result in decreased expression of E-cadherin protein. Of interest, Lei et al⁵ demonstrated a low level of expression of E-cadherin and p53, similar to our group, in their mesenchymal subgroup. Furthermore, it is worth mentioning that CDH1 and RHOA mutations were not as prevalent in the MSS/EMT subtype of ACRG compared to the TCGA.^{6, 65}

Given the amplification of gastric stem cell markers seen in the mesenchymal subtype, a role for preferential sensitivity to PI3K-AKT-mTOR inhibitors has been suggested.⁵ CDH1 and RHOA signaling pathways dysfunction has been reported in this subgroup; inhibition of RhoA activity by using an inhibitor of Rho associated kinase (ROCK) which is an effector protein of RhoA has been shown to induce apoptosis of GC cells.⁵³ A recent study demonstrated a decreased in EMT (increased expression of E-cadherin and decreased expression of N-cadherin) with danusertib (potent pan-Aurora kinase). The drug also inhibits the PI3K/Akt/mTOR-mediated signaling pathway in human GC cells.⁵⁴

Another interesting finding was the aberrant cytoplasmic expression of E-cadherin. Prior studies refer to this as “heterogeneous aberrant staining”⁵⁵ or “paranuclear staining.”⁵⁶ It has been suggested that mutation of exon 8 of CDH1 expresses an abnormal E-cadherin protein that lacks the appropriate signals for posttranslational modifications which permit the normal transport to the cell membrane and glycosylation of E-cadherin; this results in the arrest of E-cadherin in the Golgi apparatus.^{55,57} In another report, cytoplasmic E-cadherin was present in a GC with a 5-base-pair insertion in exon 9.⁵⁸ The significance of a lower aberrant p53 expression (39% vs 75%) in the two sub-clusters (cytoplasmic vs complete loss of E-cadherin) is unclear. Furthermore, there was no difference in the clinicopathologic features or survival other than a trend towards older age in the subset with cytoplasmic expression.

The cluster with aberrant p53 expression formed the majority of cases (51%), and is concordant with the CIN group (50%) of the TCGA⁶, the proliferative subtype (45%) of Leis, and the MSS/TP53 type of ACRG (35.7%)⁶⁵ A strong correlation with Lauren intestinal morphotype (81%) was seen, supporting this group's correspondence to CIN (84%)⁶, proliferative type (75%)⁵ and MSS/TP53 type (85.1%).⁶⁵ A higher lymph node stage was observed, as has been reported previously in GCs with TP53 mutations.⁵⁹⁻⁶³ A trend towards increased Her2/neu also was noted; however, the overall frequency of Her2/neu positivity was low, which may be attributed to an overall lower percentage of Lauren intestinal type cancers (49%) in our cohort, in comparison to the TOGA trial where intestinal type constituted 91% of tumors.⁶⁴

For GCs with aberrant p53, potential targets include the blockade of receptor tyrosine kinases (e.g., HER2, EGFR, vascular endothelial growth factor receptor (VEGFR), c-MET, and fibroblast growth factor receptor 2 (FGFR2) and cell cycle mediators (CCNE1, CCND1 and CDK6)), which are amplified in this group.⁶

The features of the group with normal p53 expression and increased MUC6 expression, likely correspond to the metabolic subtype reported by Lei et al.⁵ This group has been related to the spasmolytic polypeptide-expressing metaplasia (SPEM) pathway, owing to the overexpression of genes characteristic of normal gastric mucosa. Of note, the metabolic subtype is highly sensitive to 5-fluorouracil due to significantly lower expression of both thymidylate synthase (TS) and dihydropyrimidine dehydrogenase (DPD).⁵

As the prognosis of GC remains dismal despite improving surgical and adjuvant therapies, recent advances in genomic technologies have paved the way toward understanding the molecular underpinning of GCs and the identification of therapeutic biomarkers that influence outcomes and guide management strategies. However, to date, in clinical practice, GCs remain essentially classified histologically, since access and cost of high-throughput genomic technologies may limit universal molecular fingerprinting of all GCs. This study, taking into account the results of recently developed genetic and epigenetic molecular classifications of GC, demonstrated that in situ hybridization and immunohistochemical characterization of tumors can appropriately identify tumor subgroups similar to genomic profiling and could be an alternative in guiding targeted therapies.

Gastric Cancer

Malignancy of the stomach (defined as beginning at the gastroesophageal junction and ending at the duodenum) is often diagnosed at an advanced stage because early cancers typically have no associated symptoms. Diagnosis can be made based on imaging studies such as Esophagogastroduodenoscopy (EGD); double-contrast upper GI series and barium swallows; CT scanning or MRI of the chest, abdomen, and pelvis; and Endoscopic ultrasonography (EUS); and biopsy.

Standard therapies include surgical intervention and chemotherapy. Surgical interventions can include partial (subtotal) or total gastrectomy; esophagogastrectomy (e.g., for tumors of the cardia and gastroesophageal junction). Chemotherapies can include platinum-based combination chemotherapy (e.g., epirubicin/cisplatin/5-fluorouracil (5-FU); docetaxel/cisplatin/5-FU; irinotecan and cisplatin; oxaliplatin and irinotecan); trastuzumab in combination with cisplatin and capecitabine or 5-FU; and Ramucirumab following therapy with a fluoropyrimidine- or platinum-containing regimen. In addition, intraoperative radiotherapy, neoadjuvant chemotherapy, or adjuvant chemotherapy, radiotherapy, or chemoradiotherapy can be used. In cases where palliative treatment only is desired, palliative radiotherapy or surgical resections can be used.

A Five-Tier Classification of GC

In accordance with the proposed molecular classifications and based on the results of unsupervised hierarchical clustering analysis of EBV-ISH, MMR proteins, E-cadherin, and p53, a five-tier classification schema was generated; a decision tree is shown in FIG. 8.

The methods include using Epstein-Barr encoding region (EBER) in situ hybridization (EBER-ISH) to detect EBV in the tumor cells. Because of the large numbers of copies of EBERs present in latently infected cells, non-isotopic methods can be used. See, e.g., Weiss and Chen, Methods Mol Biol. 2013; 999:223-30.

The methods also include immunohistochemistry to detect DNA mismatch repair protein expression; E-cadherin expression; p53 expression; and Mucin expression in the tumor cells. Sequences of each of these proteins are known in the art; exemplary human protein sequences are provided in the following table.

| | Protein | GenBank Ref No. |
|---|---|---|
| mismatch | MLH1 MutL Homolog 1 | NP_000240.1 |
| repair | PMS2 PMS1 homolog 2, mismatch | NP_000526.2 |
| proteins | repair system component | |
| | MSH2 mutS homolog 2 | NP_000242.1 |
| | MSH6 mutS homolog 6 | NP_000170.1 |
| mucins | MUC2 mucin 2, oligomeric mucus/ | NP_002448.4 |
| | gel-forming | |
| | CDX2 caudal type homeobox 2 | NP_001256.3 |
| | MME (CD10) membrane | NP_000893.2 |
| | metalloendopeptidase | |
| | MUC5AC mucin 5AC, oligomeric | NP_001291288.1 |
| | mucus/gel-forming | |
| | MUC6 mucin 6, oligomeric mucus/ | NP_005952.2 |
| | gel-forming | |
| | P53 tumor protein p53 | NP_000537.3 |
| | CDH1 (E-cadherin) cadherin 1 | NP_001304113.1 |
| | | NP_004351.1 |

Antibodies for each of these proteins are commercially available, e.g., from Biocore, Becton Dickinson, Leica, Cell Signaling, Abcam and Ventana (From Table 2 in Setia et al.).

Included herein are methods for categorizing gastric cancer in subjects, e.g., mammalian subjects, e.g., humans or non-human veterinary or laboratory subjects. The methods rely on detection of the biomarkers described herein, e.g., EBV-ISH, MMR proteins, E-cadherin, and/or p53, to generate a five-tier classification of GC. The methods can include obtaining a sample from a subject, and evaluating the presence and/or level of the biomarkers in the sample, and comparing the presence and/or level with one or more references, e.g., a control reference that represents a normal level or expression localization of the biomarker, e.g., a level in a normal cell from an unaffected subject, and/or a disease reference that represents a level or expression localization of the proteins associated with one of the classification groups described herein. Suitable reference values can include those shown in Table 1.

As used herein the term “sample”, when referring to the material to be tested for the presence of a biological marker using the method of the invention, includes inter alia tissue known or suspected to comprise tumor cells obtained from a biopsy or tumor resection. In some embodiments, the subject has been diagnosed with gastric cancer by a method known in the art.

The presence and/or level of a protein can be evaluated using methods known in the art, e.g., using standard and quantitative immunoassay methods for proteins, including but not limited to, Western blot; enzyme linked immunosorbent assay (ELISA); biotin/avidin type assays; protein array detection; radio-immunoassay; immunohistochemistry (IHC); immune-precipitation assay; FACS (fluorescent activated cell sorting); mass spectrometry (Kim (2010) Am J Clin Pathol 134:157-162; Yasun (2012) Anal Chem 84(14):6008-6015; Brody (2010) Expert Rev Mol Diagn 10(8):1013-1022; Philips (2014) PLOS One 9(3):e90226; Pfaffe (2011) Clin Chem 57(5): 675-687). The methods typically include revealing labels such as fluorescent, chemiluminescent, radioactive, and enzymatic or dye molecules that provide a signal either directly or indirectly. As used herein, the term “label” refers to the coupling (i.e. physically linkage) of a detectable substance, such as a radioactive agent or fluorophore (e.g., phycoerythrin (PE) or indocyanine (Cy5), to an antibody or probe, as well as indirect labeling of the probe or antibody (e.g. horseradish peroxidase, HRP) by reactivity with a detectable substance.

In preferred embodiments, an IHC method may be used. IHC provides a method of detecting a biological marker in situ in structurally intact samples (i.e., wherein the sample preparation preserves the structure of the cells and subcellular compartments such as the nucleus). The presence, level and/or exact cellular location of the biological marker can be detected. Typically, a sample is fixed with formalin or paraformaldehyde, embedded in paraffin, and cut into sections for staining and subsequent inspection by confocal microscopy. Current methods of IHC use either direct or indirect labelling. The sample may also be inspected by fluorescent microscopy when immunofluorescence (IF) is performed, as a variation to IHC.

Measurement of the level of a biomarker can be direct or indirect. For example, the abundance levels of the protein can be directly quantitated, and/or compared to an internal reference, e.g., a housekeeping gene with relatively steady expression levels.

The methods can also be used to select or stratify subjects for a clinical trial, e.g., of a treatment for gastric cancer, to determine whether the treatment being tested is better for subjects with a cancer that falls in group 1, 2, 3, 4, or 5. The methods can include identifying the subjects as having a particular cancer group before administration of the treatment, or afterwards. The subjects can be selected for inclusion, or excluded from inclusion, based on the group into which their cancer call.

Treatment Selection

The method described herein can also be used for selecting, and then optionally administering, an optimal treatment for a subject. Thus the methods described herein include methods for the treatment of gastric cancer. Generally, the methods include administering a therapeutically effective amount of a treatment as described herein, to a subject who is in need of, or who has been determined to be in need of, such treatment.

As used in this context, to “treat” means to ameliorate at least one symptom of the gastric cancer. For example, a treatment can result in a reduction in tumor size, tumor growth, or metastasis or risk of metastasis.

For example, the methods can include selecting and/or administering a treatment comprising a therapeutically effective amount of a checkpoint inhibitor to a subject having a tumor in group 1. In some embodiments, the checkpoint inhibitor is an anti-PD1 or anti-PDL1 antibody, such as nivolumab, pembrolizumab, pidilizumab durvalumab, or atezolizumab. Alternatively or in addition, an immunotherapy can be used.

The methods can also include selecting and/or administering a treatment comprising a therapeutically effective amount of an immunotherapy, topoisomerase-I inhibitors, or platinum-based chemotherapy, e.g., in combination with poly (ADP-ribose) polymerase (PARP) inhibitors, to a subject having a tumor in group 2.

The methods can also include selecting and/or administering a treatment comprising a therapeutically effective amount of an inhibitor of mammalian target of rapamycin (mTOR) to a subject having a tumor in group 3.

The methods can also include selecting and/or administering a treatment comprising a therapeutically effective amount of an antimetabolite to a subject having a tumor in group 4.

The methods can also include selecting and/or administering a treatment comprising a therapeutically effective amount of an inhibitor of polo-like kinase 1 (PLK1) and/or an inhibitor of Aurora Kinase A to a subject having a tumor in group 5.

Any of the methods can also include selecting and/or administering a treatment comprising surgery or radiotherapy for any of groups 1-5.

A number of antimetabolites are known in the art, including 5-Fluorouracil (5-FU), gemcitabine, fluorouracil, cytarabine, cladribine, methotrexate, pemetrexed, capecitabine, hydroxyurea, fludarabine, pralatrexate, nelarabine, cloafarabine, decitabine, fluxuridine, and thioguanine; in preferred embodiments, the antimetabolite is a pyrimidine analog, preferably 5-Fluorouracil (5-FU), gemcitabine, fluorouracil, or cytarabine.

A number of mTOR inhibitors are known in the art, including rapamycin, ridaforolimus, temsirolimus, everolimus, sirolimus, dactolisib, AZD8055, CZ415, CC-223, voxtalisib, PI-103, KU-0063794, torkinib, ridaforolimus, Voxtalisib (SAR245409, XL765) analogue, omipalisib, OSI-027, PF-04691502, Apitolisib (GDC-0980, RG7422), GSK1059615, Gedatolisib (PF-05212384, PKI-587), WYE-354, Vistusertib (AZD2014), Torin 2, WYE-125132 (WYE-132), BGT226 (NVP-BGT226), Palomid 529 (P529), PP121, WYE-687, CH5132799, WAY-600. ETP-46464. GDC-0349, XL388, zotarolimus, MHY1485 and Chrysophanic Acid.

A number of inhibitors of polo-like kinase 1 (PLK1) are known in the art, including volasertib, BI 2536, GSK461364, NMS-P937 (NMS1286937), Ro3280, MLN0905, SBE 13 HCl, HMN-214, HMN-176, and rigosertib. A number of inhibitors of Aurora Kinase A are known in the art, including MK-5108 (VX-689), MK-8745, Alisertib (MLN8237), Aurora A Inhibitor I, MLN8054, ENMD-2076 L-(+)-Tartaric acid, ENMD-2076, KW-2449, VX-680 (Tozasertib, MK-0457), PF-03814735, AT9283, AMG-900, CYC116, SNS-314 Mesylate, JNJ-7706621, Reversine, Danusertib (PHA-739358), CCT137690, TAK-901, PHA-680632, CCT129202, ZM 447439, GSK1070916, Barasertib (AZD1152-HQPA), alisertib (MLN8327), or Hesperadin.

Immunotherapies include those therapies that target tumor-induced immune suppression; see, e.g., Stewart and Smyth, Cancer Metastasis Rev. 2011 March; 30(1):125-40. In some embodiments, these therapies may primarily target immunoregulatory cell types such as regulatory T cells (Tregs) or M2 polarized macrophages, e.g., by reducing number, altering function, or preventing tumor localization of the immunoregulatory cell types. For example, Treg-targeted therapy includes anti-GITR monoclonal antibody (TRX518), cyclophosphamide (e.g., metronomic doses), arsenic trioxide, paclitaxel, sunitinib, oxaliplatin, PLX4720, anthracycline-based chemotherapy, Daclizumab (anti-CD25); Immunotoxin eg. Ontak (denileukin diftitox); lymphoablation (e.g., chemical or radiation lymphoablation) and agents that selectively target the VEGF-VEGFR signaling axis, such as VEGF blocking antibodies (e.g., bevacizumab), or inhibitors of VEGFR tyrosine kinase activity (e.g., lenvatinib) or ATP hydrolysis (e.g., using ectonucleotidase inhibitors, e.g., ARL67156 (6-N,N-Diethyl-D-β,γ-dibromomethyleneATP trisodium salt), 8-(4-chlorophenylthio) cAMP (pCPT-cAMP) and a related cyclic nucleotide analog (844-chlorophenylthio] cGMP; pCPT-cGMP) and those described in WO 2007135195, as well as mAbs against CD73 or CD39). Docetaxel also has effects on M2 macrophages. See, e.g., Zitvogel et al., Immunity 39:74-88 (2013). In another example, M2 macrophage targeted therapy includes clodronate-liposomes (Zeisberger, et al., Br J Cancer. 95:272-281 (2006)), DNA based vaccines (Luo, et al., J Clin Invest. 116(8): 2132-2141 (2006)), and M2 macrophage targeted pro-apoptotic peptides (Cieslewicz, et al., PNAS. 110(40): 15919-15924 (2013)). Immnotherapies that target Natural Killer T (NKT) cells can also be used, e.g., that support type I NKT over type II NKT (e.g., CD1d type I agonist ligands) or that inhibit the immune-suppressive functions of NKT, e.g., that antagonize TGF-beta or neutralize CD1d.

Some useful immunotherapies target the metabolic processes of immunity, and include adenosine receptor antagonists and small molecule inhibitors, e.g., istradefylline (KW-6002) and SCH-58261; indoleamine 2,3-dioxygenase (IDO) inhibitors, e.g., Small molecule inhibitors (e.g., 1-methyl-tryptophan (1MT), 1-methyl-d-tryptophan (D1MT), and Toho-1) or IDO-specific siRNAs, or natural products (e.g., Brassinin or exiguamine) (see, e.g., Munn, Front Biosci (Elite Ed). 2012 Jan. 1; 4:734-45) or monoclonal antibodies that neutralize the metabolites of IDO, e.g., mAbs against N-formyl-kynurenine.

In some embodiments, an immunotherapy may antagonize the action of cytokines and chemokines such as IL-10, TGF-beta, IL-6, CCL2 and others that are associated with immunosuppression in cancer. For example, TGF-beta neutralizing therapies include anti-TGF-beta antibodies (e.g. fresolimumab, Infliximab, Lerdelimumab, GC-1008), antisense oligodeoxynucleotides (e.g., Trabedersen), and small molecule inhibitors of TGF-beta (e.g. LY2157299), (Wojtowicz-Praga, Invest New Drugs. 21(1): 21-32 (2003)). Another example of therapies that antagonize immunosuppression cytokines can include anti-IL-6 antibodies (e.g. siltuximab) (Guo, et al., Cancer Treat Rev. 38(7):904-910 (2012). mAbs against IL-10 or its receptor can also be used, e.g., humanized versions of those described in Llorente et al., Arthritis & Rheumatism, 43(8): 1790-1800, 2000 (anti-IL-10 mAb), or Newton et al., Clin Exp Immunol. 2014 July; 177(1):261-8 (Anti-interleukin-10R1 monoclonal antibody). mAbs against CCL2 or its receptors can also be used. In some embodiments, the cytokine immunotherapy is combined with a commonly used chemotherapeutic agent (e.g., gemcitabine, docetaxel, cisplatin, tamoxifen) as described in U.S. Pat. No. 8,476,246.

In some embodiments, immunotherapies can include agents that are believed to elicit “danger” signals, e.g., “PAMPs” (pathogen-associated molecular patterns) or “DAMPs” (damage-associated molecular patterns) that stimulate an immune response against the cancer. See, e.g., Pradeu and Cooper, Front Immunol. 2012, 3:287; Escamilla-Tilch et al., Immunol Cell Biol. 2013 November-December; 91(10):601-10. In some embodiments, immunotherapies can agonize toll-like receptors (TLRs) to stimulate an immune response. For example, TLR agonists include vaccine adjuvants (e.g., 3M-052) and small molecules (e.g., Imiquimod, muramyl dipeptide, CpG, and mifamurtide (muramyl tripeptide)) as well as polysaccharide krestin and endotoxin. See, Galluzi et al., Oncoimmunol. 1(5): 699-716 (2012), Lu et al., Clin Cancer Res. Jan. 1, 2011; 17(1): 67-76, U.S. Pat. Nos. 8,795,678 and 8,790,655. In some embodiments, immunotherapies can involve administration of cytokines that elicit an anti-cancer immune response, see Lee & Margolin, Cancers. 3: 3856-3893(2011). For example, the cytokine IL-12 can be administered (Portielje, et al., Cancer Immunol Immunother. 52: 133-144 (2003)) or as gene therapy (Melero, et al., Trends Immunol. 22(3): 113-115 (2001)). In another example, interferons (IFNs), e.g., IFNgamma, can be administered as adjuvant therapy (Dunn et al., Nat Rev Immunol. 6: 836-848 (2006)).

In some embodiments, immunotherapies can antagonize cell surface receptors to enhance the anti-cancer immune response. For example, antagonistic monoclonal antibodies that boost the anti-cancer immune response can include antibodies that target CTLA-4 (ipilimumab, see Tarhini and Iqbal, Onco Targets Ther. 3:15-25 (2010) and U.S. Pat. No. 7,741,345 or Tremelimumab) or antibodies that target PD-1 (nivolumab, see Topalian, et al., NEJM. 366(26): 2443-2454 (2012) and WO2013/173223A1, pembrolizumab/MK-3475, Pidilizumab (CT-011)).

Some immunotherapies enhance T cell recruitment to the tumor site (such as Endothelin receptor-A/B (ETRA/B) blockade, e.g., with macitentan or the combination of the ETRA and ETRB antagonists BQ123 and BQ788, see Coffman et al., Cancer Biol Ther. 2013 February; 14(2):184-92), or enhance CD8 T-cell memory cell formation (e.g., using rapamycin and metformin, see, e.g., Pearce et al., Nature. 2009 Jul. 2; 460(7251):103-7; Mineharu et al., Mol Cancer Ther. 2014 Sep. 25. pii: molcanther.0400.2014; and Berezhnoy et al., Oncoimmunology. 2014 May 14; 3:e28811). Immunotherapies can also include administering one or more of: adoptive cell transfer (ACT) involving transfer of ex vivo expanded autologous or allogeneic tumor-reactive lymphocytes, e.g., dendritic cells or peptides with adjuvant; cancer vaccines such as DNA-based vaccines, cytokines (e.g., IL-2), cyclophosphamide, anti-interleukin-2R immunotoxins, Prostaglandin E2 Inhibitors (e.g., using SC-50) and/or checkpoint inhibitors including antibodies such as anti-CD137 (BMS-663513), anti-PD1 (e.g., Nivolumab, pembrolizumab/MK-3475, Pidilizumab (CT-011)), anti-PDL1 (e.g., BMS-936559, MPDL3280A), or anti-CTLA-4 (e.g., ipilumimab; see, e.g., Kruger et al., “Immune based therapies in cancer,” Histol Histopathol. 2007 June; 22(6):687-96; Eggermont et al., “Anti-CTLA-4 antibody adjuvant therapy in melanoma,” Semin Oncol. 2010 October; 37(5):455-9; Klinke D J 2nd, “A multiscale systems perspective on cancer, immunotherapy, and Interleukin-12,” Mol Cancer. 2010 Sep. 15; 9:242; Alexandrescu et al., “Immunotherapy for melanoma: current status and perspectives,” J Immunother. 2010 July-August; 33(6):570-90; Moschella et al., “Combination strategies for enhancing the efficacy of immunotherapy in cancer patients,” Ann N Y Acad Sci. 2010 April; 1194:169-78; Ganesan and Bakhshi, “Systemic therapy for melanoma,” Natl Med J India. 2010 January-February; 23(1):21-7; Golovina and Vonderheide, “Regulatory T cells: overcoming suppression of T-cell immunity,” Cancer J. 2010 July-August; 16(4):342-7. In some embodiments, the methods include administering a composition comprising tumor-pulsed dendritic cells, e.g., as described in WO2009/114547 and references cited therein. See also Shiao et al., Genes & Dev. 2011. 25: 2559-2572.

Topoisomerase-I inhibitors include indenoisoquinolines such as irinotecan, indotecan and indimitecan, and camptothecins. See, e.g., Pommier, Nature Reviews Cancer 6, 789-802 (October 2006).

Inhibitors of the enzyme poly ADP ribose polymerase (PARP) can include niraparib (MK-4827), olaparib, veliparib, talazoparib, recuparib, CEP 9722, BGB-290, and E7016, among others.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### EXAMPLES

The invention is further described in the following examples, which do not limit the scope of the invention described in the claims.

Materials and Methods

The following materials and methods were used in the Examples set forth below.

Tissue samples. The tissue microarrays (TMA) (24 cores per slide, 3 mm each, 1-2 cores/case with 2 controls per slide) were constructed from 146 primary GC resections performed at Massachusetts General Hospital from 1988 to 2007. The review board at Massachusetts General Hospital approved this study. Collected clinical and pathologic features include age ((greater than or less than 70 years of age, using 70 years (median age)), gender, maximal size ((greater than or less than 5 cm, using 5 cm (median)), Lauren type (diffuse, intestinal), TNM stage, tumor grade (grades 1/2/3/4),⁷ tumor site (cardia, body/fundus, antrum, multifocal), lymphovascular and/or venous invasion, perineural invasion and overall survival (months). At least two gastrointestinal pathologists reviewed all H&E slides.

Biomarker panel. Expression for 14 different biomarkers was performed and included EBER in situ hybridization (ISH), p53, MMR proteins (MLH1, PMS2, MSH2, and MSH6), E-Cadherin, PD-L1, MUC2, CDX2, CD10, MUCSAC, MUC6, and HER2. The specifications and dilution, site (membranous, nuclear, and cytoplasmic), scoring criteria, and pattern of staining for all biomarkers are listed in Table 1. Appropriate RNA controls, to assure RNA preservation, were performed on all TMA slides where required. Two gastrointestinal pathologists reviewed all immunohistochemical stains and in situ hybridization (GYL and NS).

TABLE 1
Diagnostic biomarkers. This table shows the biomarkers used in the study with details including the antibody
specifications, dilution, scoring criteria, and aberrant pattern of staining.

| | Clone and | | | Score & | | Interpreted |
|---|---|---|---|---|---|---|
| Biomarker | dilution | Cell type | Site | Criteria | Column1 | as aberrant |
| EBER-ISH | Leica, NA† | Tumor | Nucleus | 0 | Absent | N |
| | RTU* | | | 1 | Present | Y |
| MLH1 | Biocore, E305 | Tumor | Nucleus | 0 | Absent | Y |
| | 1:50 | | | 1 | Present | N |
| PMS2 | BD MOR4g | Tumor | Nucleus | 0 | Absent | Y |
| | 0.111111111 | | | 1 | Present | N |
| MSH2 | Biocore, 25D12 | Tumor | Nucleus | 0 | Absent | Y |
| | 1:25 | | | 1 | Present | N |
| MSH6 | Biocore 44 | Tumor | Nucleus | 0 | Absent | Y |
| | 1:25 | | | 1 | Present | N |
| P53 | Leica,DO-7 | Tumor | Nucleus | 0 | Complete loss | Y |
| | RTU* | | | 0.5 | Weak, patchy | N |
| | | | | 1 | Diffuse, strong | Y |
| E-cadherin | Leica, 36B5 | Tumor | Cytoplasmic and | 0 | Complete loss | Y |
| | | | membranous | | | |
| | RTU* | | | 0.33 | Cytoplasmic | Y |
| | | | | 0.66 | Cytoplasmic and | N |
| | | | | | Membranous | |
| | | | | 0.99 | Membranous | N |
| PD-L1 | Cell Signaling | Tumor and | Cytoplasmic and | 0 | Absent | N |
| | EIL3N, 1:200 | macrophages | membranous | | | |
| | | | | 0.5 | Cytoplasmic or membranous | N |
| | | | | | expression in macrophages | |
| | | | | 1 | Membranous in tumor cells | Y |
| MUC2 | Leica, COP58 | Tumor | Cytoplasmic | 0 | <10% cells positive | N |
| | 0.180555556 | | | 1 | 10-25% cells positive | Y |
| | | | | 2 | >25% cells positive | Y |
| MUC5AC | Leica CCH2, | Tumor | Cytoplasmic | 0 | <10% cells positive | N |
| | 0.111111111 | | | 1 | 10-25% cells positive | Y |
| | | | | 2 | >25% cells positive | Y |
| MUC6 | Leica CLH5 | Tumor | Cytoplasmic | 0 | <10% cells positive | N |
| | 0.111111111 | | | 1 | 10-25% cells positive | Y |
| | | | | 2 | >25% cells positive | Y |
| CDX2 | Abcam AMT28 | Tumor | Nuclear | 0 | <10% cells positive | N |
| | 1:25 | | | 1 | 10-25% cells positive | Y |
| | | | | 2 | >25% cells positive | Y |
| CD10 | Leica 56C6 | Tumor | Membranous | 0 | <10% cells positive | N |
| | RTU* | | | 1 | 10-25% cells positive | Y |
| | | | | 2 | >25% cells positive | Y |
| Her2 | Ventana SP3 | Tumor | Membranous | 0 | No reactivity, or | N |
| | | | | | membranous reactivity in <5 | |
| | | | | | clustered tumor cells | |
| | 1:08 | | | 1 | Tumor cell cluster (≥5 cells) | N |
| | | | | | with a faint/barely | |
| | | | | | perceptible membranous | |
| | | | | | reactivity | |
| | | | | 2 | Weak-to-moderate | N |
| | | | | | complete, basolateral or | |
| | | | | | lateral membranous | |
| | | | | | reactivity in tumor cell | |
| | | | | | cluster (≥5 cells) | |
| | | | | 3 | Tumor cell cluster (≥5 cells) | Y |
| | | | | | with a strong complete, | |
| | | | | | basolateral or lateral | |
| | | | | | membranous staining | |

*RTU Ready to use
†NA not available

Rationale for biomarker evaluation. EBER-ISH is the gold standard to detect EBV status, and localizes the abundantly expressed long noncoding RNAs EBER1 or EBER2 in malignant cells.^{8,9} Use of immunohistochemistry for MMR proteins has a high sensitivity, specificity, and positive and negative predictive value for deficiency of the MMR system.¹⁰ Additionally, the predominant mechanism of MSI in GC is promoter hypermethylation of MLH1 rather than mutations.^{6,11} Several studies have shown an association between aberrant E-cadherin expression and diffuse-type adenocarcinomas,^{12,13} and it has been suggested that loss of E-cadherin is a phenotypic expression of the genetic alteration noted in diffuse type GC (CDH1 mutations).¹⁴ Immunohistochemical staining of E-cadherin has been shown to mirror the mRNA expression also.¹⁵ A disconcordance between the immunostaining of p53 and its mutational status has been reported by several studies;¹⁶ however, the difference has been reported to decrease if the criteria for overexpression are stringently applied,¹⁷ as in the current study (Table 1).

Statistics. The statistical analysis was performed using R software for statistical computing v3.0.2 (r-project.org). Categorical variables were compared with the Fisher Exact Test, and P value of <0.05 (two-sided) was considered statistically significant. The overall survival was measured from the date of resection of the GC to the date of death from any cause (recorded from the medical chart and/or the Social Security death index, genealogybank.com/gbnk/ssdi/). The survival data was analyzed by Cox Proportional Hazards analysis. Unsupervised hierarchical clustering analysis with average linkage algorithms was applied to the dataset with the chosen 5 biomarkers (EBER-ISH, MLH1, PMS2, E-cadherin, and p53), followed by comparison of clinical phenotype and outcome analysis. This was performed using GeneCluster 3.0 (eisenlab.org/eisen/?page_id=42); TreeView (rana.lbl.gov/EisenSoftware.htm) was used for graphical representation of the results.

### Example 1. Clinicopathological Features of the Five Subtypes of Gastric Cancers

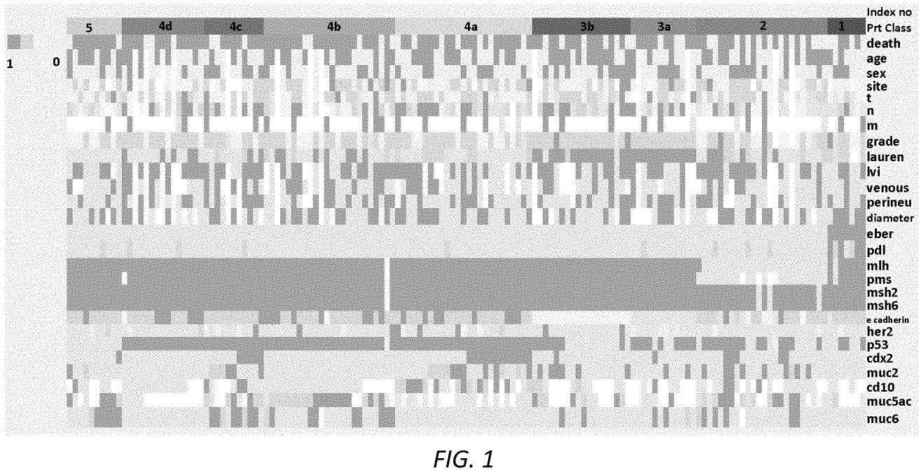

In accordance with the proposed molecular classifications and based on the results of unsupervised hierarchical clustering analysis of the expression EBV-ISH, MMR proteins, E-cadherin, and p53, a five-tier classification algorithm of GC was generated. Out of the cohort of 146 cases, EBER-positive-GC constituted 5% of the cases (n=7) (Cluster 1); MMR deficient-GC represented 16% of the cases (n=24) (Cluster 2); aberrant expression of E-cadherin (Cluster 3) was noted in 21% of the GCs (n=30); and GC with aberrant p53 expression (Cluster 4) represented 51% of the cases (n=71). Cluster 4 with aberrant p53 expression was further subclassified into intestinal (33%, 25/75, MUC2 and/or CD10 positive), gastric (32%, 24/75, MUCSAC and/or MUC6 positive), mixed (15%, 11/75, both MUC2 and/or CD10 positive and MUCSAC and/or MUC6 positive), and null (20%, 15/75, MUC2, CD10, MUCSAC and MUC6 negative) sub-clusters. The remaining cases with normal p53 expression were designated as Cluster 5, comprising 7% of GCs (n=10). FIG. 1 depicts the biomarker expression-based clusters along with key clinicopathologic features. FIG. 8 presents the protein based expression.

EBV-Positive GC (Cluster 1):

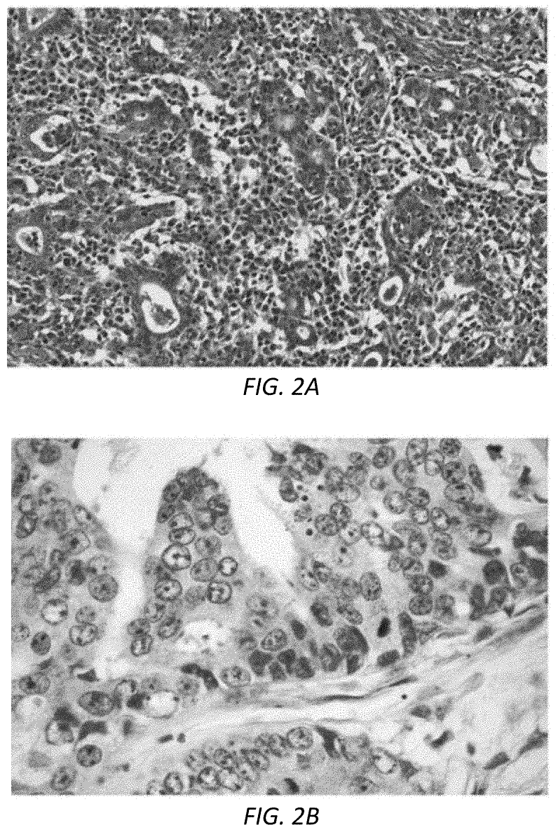

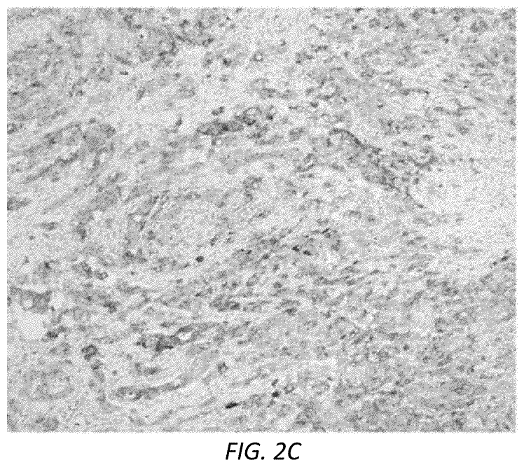

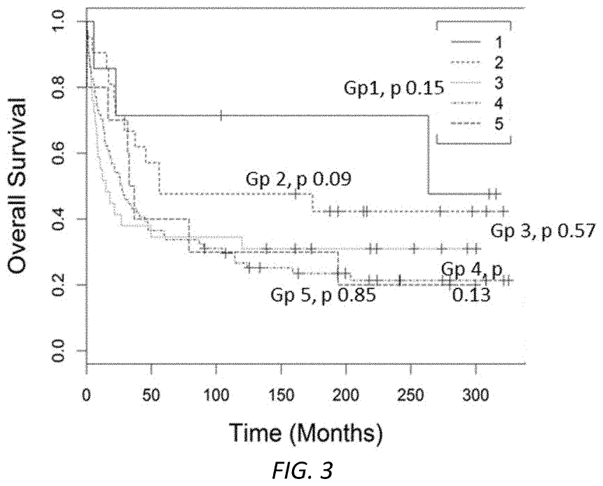

See clinical characteristics in Table 2. The lesions were characterized by prominent lymphoid infiltrate in all cases. (FIG. 2) EBV-positive GCs trended towards a better survival (p 0.15, CI 0.14-1.36, HR 0.43 median survival 263.51 months vs. 29.31 months for non-EBV GC, FIG. 3); however, the difference was not statistically significant. A strong correlation with membranous expression of PD-L1 in tumor cells was seen (57% vs 0%, p-0.001) (FIG. 2).

TABLE 2
Clinicopathologic characteristics of the various gastric cancer subtypes.

| | Mean age | M:F | |
|---|---|---|---|
| | (in years) | ratio | Site (ratio) |
| Epstein Barr Virus | 64.9 ± 9.8 | 1.3:1 | antrum non antrum: 0:7¹ |
| positive gastric cancers | | | |
| Microsatellite unstable | 68 ± 14.8 | 1.3:1 | cardia: non cardia: 1 5.3² |
| gastric cancers | | | |
| Gastric cancers with | 67.2 ± 14.9 | 1.6:1 | cardia : no cardia. 1:3³ |
| aberrant E-cadherin expression | | | |
| Gastric cancers with | 68.3 ± 12.2 | 1.5:1 | antrum: non-antrum: 1:1 2⁴ |
| aberrant p53 expression | | | |
| Gastric cancers with | 65.5 ± 15.5 | 2.3:1 | body: non-body: 1:1⁵ |
| normal p53 expression | | | |

¹: the ratio was 1:1.15 in the rest of the cohort (p = 0.02)
²: the ratio was 1:4.05 in the rest of the cohort (p = 0.762)
³: the ratio was 1:4.5 in the rest of the cohort (p = 0.053)
⁴: the ratio was 1:1.29 in the rest of the cohort (p = 0.85)
⁵: the ratio was 1:2.6 in the rest of the cohort (p = 0.287)

MSI Gastric Cancers (Cluster 2):

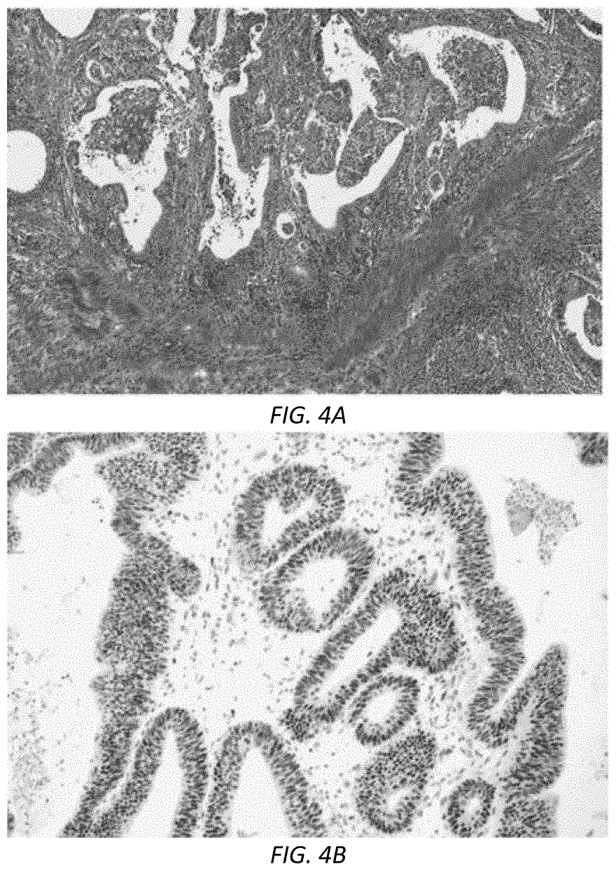

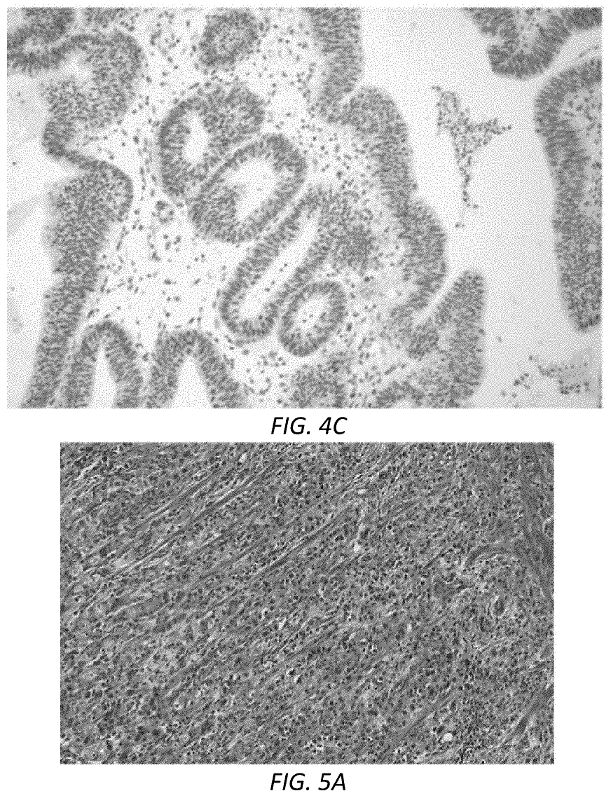

See clinical characteristics in Table 2. There was a significant association with MUC2 expression (8/24 (33%) in this cluster compared to the rest of the cohort 18/122 (15%), p 0.04). This cluster had a lower frequency of lymph node (LN) metastasis (LN stage category >N1, 27% MSI GC vs. 55% non-MSI GC, p 0.02, CI 0.09-0.91), and the patients trended toward better survival (p 0.09, CI 0.32-1.09, HR 0.59, median survival-56.05 months vs 27.12 months for non-MSI GC, FIG. 3). The loss of MLH1 and PMS2 was the predominant pattern (95.8%); an additional case showed loss of PMS2 only (4.16%) (FIG. 4)

Gastric Cancers with Aberrant E-Cadherin Expression (Cluster 3):

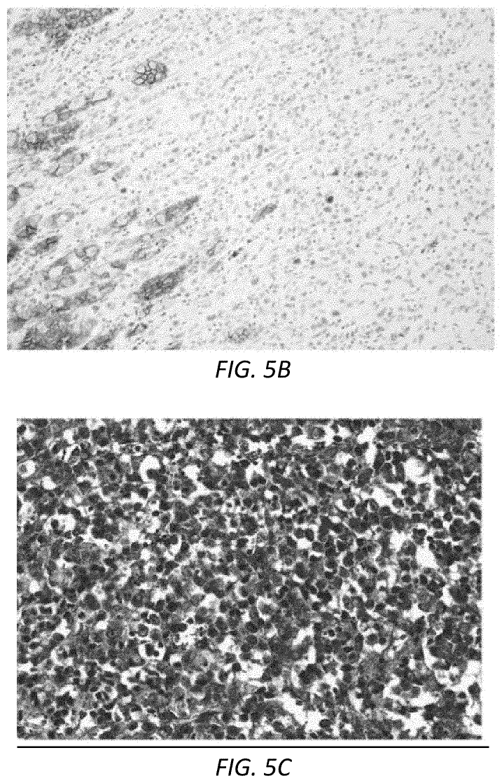

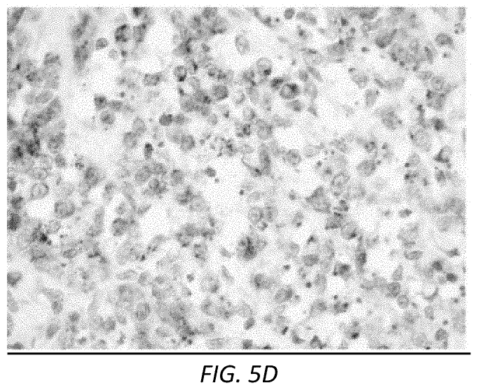

See clinical characteristics in Table 2. The GCs were predominantly of the poorly cohesive (i.e., diffuse) type (27/30, 90%). This group was subdivided into Cluster 3A showing complete loss of E-cadherin (40%) and Cluster 3B with cytoplasmic granular E-cadherin (60%) staining (FIG. 5). The patients of Cluster 3 had significantly lower aberrant p53 expression (16/30, 53%) versus 91/114 (80%) in the remainder (p 0.004). The difference was more significant in subcluster 3B versus the remainder ((7/18, 39%) in 3B versus 100/126 (79%) in the remainder, p 0.0007)). The patients in 3B also were older than 3A (3A: 62.4 yrs±16.69, 30%>70 yrs; 3B: 71.4 yrs±11.86, 65%>70 yrs). There was no survival difference (p 0.57, CI 0.71-1.88, HR 1.15, median survival—14.86 months vs 35.79 months for the remaining GC cases, FIG. 3) in this group.

Gastric Cancers with Aberrant p53 Expression (Cluster 4)

See clinical characteristics in Table 2. The tumors were predominantly of intestinal type (61/76, 81%). The lesions were associated with higher lymph node stage >N0 (p 0.03, CI 1.06-6.24, 81% vs. 63% in the remaining GCs). This cluster trended towards increased Her2 staining (Her2 >0, p 0.05, CI 0.97-11.34, 20% vs. 8% in the remaining GC cases). No significant survival difference was noted in this group (p 0.13 CI 0.92-2.03, HR 1.36, median survival 26.84 months vs 37.55 months in the remaining GC cases, FIG. 3).

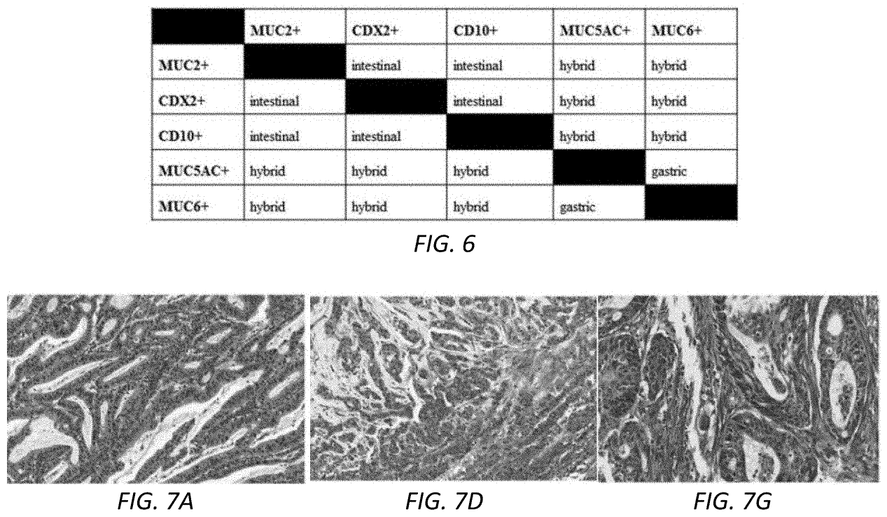

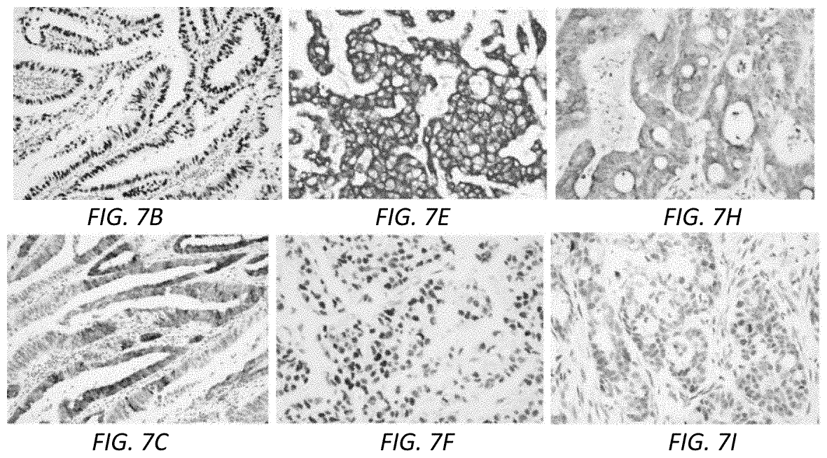

Based on the pattern of MUC and CD10 expression, this cluster was subdivided into 4 subgroups (intestinal (MUC2±CD10); gastric (MUCSAC MUC6); mixed (MUC2±CD10 AND MUCSAC±MUC6); and null (MUC2-CD10-, MUCSAC-, and MUC6-) subgroups) (FIGS. 6 and 7). Intestinal and mixed GCs trended towards increased Her2/neu expression ((Intestinal GC: Her2 >0, p 0.10, CI 0.60-13.40, 17% vs. 6%; mixed GC: Her2 >0, p 0.05, CI 0.76-17.70, 36% vs. 13%)). Null GC were associated with higher frequency of nodal metastasis >N0 (p 0.03, CI 1.03->10³, 100% vs. 69% in the remaining cases). Those were also associated with a significantly higher percentage of lymphovascular invasion (p 0.01, CI 1.35->10³, 100% vs. 61%). There was no survival difference between the sub-categories.

Gastric Cancers with Normal p53 Expression (Cluster 5)

The remaining cancers (7% of cases with normal p53 expression) constituted Cluster 5. Observed in this group were a lack of EBV-ISH, MMR deficiency and aberrant E-cadherin expression. See clinical characteristics in Table 2. Morphologically, all 10 cases presented a gland-forming (i.e., intestinal) morphotype. This cluster was associated with an increased expression of MUC6 (p 0.01, CI 1.23-28.88, 60% vs. 21%). There was no survival difference (p 0.84, CI 0.52-2.22, HR 1.08, median survival 34.85 months vs 29.23 months for the remaining GC cases. (FIG. 3).

### REFERENCES

1. Surveillance, epidemiology, and end results (SEER) program (seer.cancer.gov) research data (1973-2012), national cancer institute, DCCPS, surveillance research program, surveillance systems branch, released April 2015, based on the November 2014 submission.
2. Lauren P. The two histological main types of gastric carcinoma: Diffuse and so-called intestinal-type carcinoma. an attempt at a histo-clinical classification. Acta Pathol Microbiol Scand. 1965; 64:31-49.
3. Lauwers G Y, Carneiro F, Graham D Y, et al. Gastric carcinoma. In: Bosman F T, Carneiro F, Hruban R H, Theise N D, eds. WHO classification of tumours of the digestive system. 4th ed. Lyon: IARC Press; 2010:48-58.
4. Sunakawa Y, Lenz H J. Molecular classification of gastric adenocarcinoma: Translating new insights from the cancer genome atlas research network. Curr Treat Options Oncol. 2015; 16(4):331-015-0331-y.
5. Lei Z, Tan I B, Das K, et al. Identification of molecular subtypes of gastric cancer with different responses to PI3-kinase inhibitors and 5-fluorouracil. Gastroenterology. 2013; 145(3):554-565.
6. Cancer Genome Atlas Research Network. Comprehensive molecular characterization of gastric adenocarcinoma. Nature. 2014; 513(7517):202-209.
7. Edge S B, Byrd D R, Carducci M A, Compton C C, eds. AJCC cancer staging manual. 7th ed. New York, N.Y.: Springer; 2009.
8. Shinozaki-Ushiku A, Kunita A, Fukayama M. Update on epstein-barr virus and gastric cancer (review). Int J Oncol. 2015; 46(4):1421-1434.
9. Gulley M L, Tang W. Laboratory assays for epstein-barr virus-related disease. J Mol Diagn. 2008; 10(4):279-292.
10. Chiaravalli A M, Cornaggia M, Furlan D, et al. The role of histological investigation in prognostic evaluation of advanced gastric cancer. analysis of histological structure and molecular changes compared with invasive pattern and stage. Virchows Arch. 2001; 439(2):158-169.
11. Pinto M, Wu Y, Mensink R G, Cirnes L, Seruca R, Hofstra R M. Somatic mutations in mismatch repair genes in sporadic gastric carcinomas are not a cause but a consequence of the mutator phenotype. Cancer Genet Cytogenet. 2008; 180(2):110-114.
12. Gabbert H E, Mueller W, Schneiders A, et al. Prognostic value of E-cadherin expression in 413 gastric carcinomas. Int J Cancer. 1996; 69(3):184-189.
13. Blok P, Craanen M E, Dekker W, Tytgat G N. Loss of E-cadherin expression in early gastric cancer. Histopathology. 1999; 34(5):410-415.
14. Carneiro F, Machado J C, Seruca R, Sobrinho-Simoes M. E-cadherin changes in gastric carcimona. Histopathology. 1999; 35(5):477-478.
15. Rosivatz E, Becker I, Specht K, et al. Differential expression of the epithelial-mesenchymal transition regulators snail, SIP1, and twist in gastric cancer. Am J Pathol. 2002; 161(5):1881-1891.
16. Fenoglio-Preiser C M, Wang J, Stemmermann G N, Noffsinger A. TP53 and gastric carcinoma: A review. Hum Mutat. 2003; 21(3):258-270.
17. Ando K, Oki E, Saeki H, et al. Discrimination of p53 immunohistochemistry-positive tumors by its staining pattern in gastric cancer. Cancer Med. 2015; 4(1):75-83.
18. Truong C D, Feng W, Li W, et al. Characteristics of epstein-barr virus-associated gastric cancer: A study of 235 cases at a comprehensive cancer center in U.S.A. J Exp Clin Cancer Res. 2009; 28:14-9966-28-14.
19. Geddert H, zur Hausen A, Gabbert H E, Sarbia M. EBV-infection in cardiac and non-cardiac gastric adenocarcinomas is associated with promoter methylation of p16, p14 and APC, but not hMLH1. Cell Oncol (Dordr). 2011; 34(3):209-214.
20. Sudo M, Chong J M, Sakuma K, et al. Promoter hypermethylation of E-cadherin and its abnormal expression in epstein-barr virus-associated gastric carcinoma. Int J Cancer. 2004; 109(2):194-199.
21. Lee H S, Chang M S, Yang H K, Lee B L, Kim W H. Epstein-barr virus-positive gastric carcinoma has a distinct protein expression profile in comparison with epstein-barr virus-negative carcinoma. Clin Cancer Res. 2004; 10(5):1698-1705.
22. Zhao J, Liang Q, Cheung K F, et al. Genome-wide identification of epstein-barr virus-driven promoter methylation profiles of human genes in gastric cancer cells. Cancer. 2013; 119(2):304-312.
23. Hino R, Uozaki H, Inoue Y, et al. Survival advantage of EBV-associated gastric carcinoma: Survivin up-regulation by viral latent membrane protein 2A. Cancer Res. 2008; 68(5):1427-1435.
24. Gulley M L. Genomic assays for epstein-barr virus-positive gastric adenocarcinoma. Exp Mol Med. 2015; 47:e134.
25. Gulley M L, Pulitzer D R, Eagan P A, Schneider B G. Epstein-barr virus infection is an early event in gastric carcinogenesis and is independent of bcl-2 expression and p53 accumulation. Hum Pathol. 1996; 27(1):20-27.
26. Kim S Y, Park C, Kim H J, et al. Deregulation of immune response genes in patients with epstein-barr virus-associated gastric cancer and outcomes. Gastroenterology. 2015; 148(1):137-147.e9.
27. Grogg K L, Lohse C M, Pankratz V S, Halling K C, Smyrk T C.

Lymphocyte-rich gastric cancer: Associations with epstein-barr virus, microsatellite instability, histology, and survival. Mod Pathol. 2003; 16(7):641-651.
28. Zazula M, Ferreira A M, Czopek J P, et al. CDH1 gene promoter hypermethylation in gastric cancer: Relationship to goseki grading, microsatellite instability status, and EBV invasion. Diagn Mol Pathol. 2006; 15(1):24-29.
29. Kusano M, Toyota M, Suzuki H, et al. Genetic, epigenetic, and clinicopathologic features of gastric carcinomas with the CpG island methylator phenotype and an association with epstein-barr virus. Cancer. 2006; 106(7):1467-1479.
30. Chang M S, Lee H S, Kim C W, Kim Y I, Kim W H. Clinicopathologic characteristics of epstein-barr virus-incorporated gastric cancers in korea. Pathol Res Pract. 2001; 197(6):395-400.
31. Li S, Du H, Wang Z, Zhou L, Zhao X, Zeng Y. Meta-analysis of the relationship between epstein-barr virus infection and clinicopathological features of patients with gastric carcinoma. Sci China Life Sci. 2010; 53(4):524-530.
32. Murphy G, Pfeiffer R, Camargo M C, Rabkin C S. Meta-analysis shows that prevalence of epstein-barr virus-positive gastric cancer differs based on sex and anatomic location. Gastroenterology. 2009; 137(3):824-833.
33. Camargo M C, Kim W H, Chiaravalli A M, et al. Improved survival of gastric cancer with tumour epstein-barr virus positivity: An international pooled analysis. Gut. 2014; 63(2):236-243.
34. Cheng N, Hui D Y, Liu Y, et al. Is gastric lymphoepithelioma-like carcinoma a special subtype of EBV-associated gastric carcinoma? new insight based on clinicopathological features and EBV genome polymorphisms. Gastric Cancer. 2015; 18(2):246-255.
35. Song H J, Srivastava A, Lee J, et al. Host inflammatory response predicts survival of patients with epstein-barr virus-associated gastric carcinoma. Gastroenterology. 2010; 139(1):84-92.e2.
36. van Beek J, zur Hausen A, Klein Kranenbarg E, et al. EBV-positive gastric adenocarcinomas: A distinct clinicopathologic entity with a low frequency of lymph node involvement. J Clin Oncol. 2004; 22(4):664-670.
37. Park S, Choi M G, Kim K M, et al. Lymphoepithelioma-like carcinoma: A distinct type of gastric cancer. J Surg Res. 2015; 194(2):458-463.
38. Arai T, Sakurai U, Sawabe M, et al. Frequent microsatellite instability in papillary and solid-type, poorly differentiated adenocarcinomas of the stomach. Gastric Cancer. 2013; 16(4):505-512.
39. Wang K, Yuen S T, Xu J, et al. Whole-genome sequencing and comprehensive molecular profiling identify new driver mutations in gastric cancer. Nat Genet. 2014; 46(6):573-582.
40. Chung Y J, Kim K M, Choi J R, Choi S W, Rhyu M G. Relationship between intratumor histological heterogeneity and genetic abnormalities in gastric carcinoma with microsatellite instability. Int J Cancer. 1999; 82(6):782-788.
41. Lee H S, Choi S I, Lee H K, et al. Distinct clinical features and outcomes of gastric cancers with microsatellite instability. Mod Pathol. 2002; 15(6):632-640.
42. Seo H M, Chang Y S, Joo S H, et al. Clinicopathologic characteristics and outcomes of gastric cancers with the MSI-H phenotype. J Surg Oncol. 2009; 99(3):143-147.
43. Gu M, Kim D, Bae Y, Choi J, Kim S, Song S. Analysis of microsatellite instability, protein expression and methylation status of hMLH1 and hMSH2 genes in gastric carcinomas. Hepatogastroenterology. 2009; 56(91-92):899-904.
44. Corso G, Pedrazzani C, Marrelli D, Pascale V, Pinto E, Roviello F. Correlation of microsatellite instability at multiple loci with long-term survival in advanced gastric carcinoma. Arch Surg. 2009; 144(8):722-727.
45. Beghelli S, de Manzoni G, Barbi S, et al. Microsatellite instability in gastric cancer is associated with better prognosis in only stage I I cancers. Surgery. 2006; 139(3):347-356.
46. Leung S Y, Yuen S T, Chung L P, Chu K M, Chan A S, Ho J C. hMLH1 promoter methylation and lack of hMLH1 expression in sporadic gastric carcinomas with high-frequency microsatellite instability. Cancer Res. 1999; 59(1):159-164.
47. Hudler P. Genetic aspects of gastric cancer instability. ScientificWorldJournal. 2012; 2012:761909.
48. Ribic C M, Sargent D J, Moore M J, et al. Tumor microsatellite-instability status as a predictor of benefit from fluorouracil-based adjuvant chemotherapy for colon cancer. N Engl J Med. 2003; 349(3):247-257.
49. Liu X P, Tsushimi K, Tsushimi M, et al. Expression of p53 protein as a prognostic indicator of reduced survival time in diffuse-type gastric carcinoma. Pathol Int. 2001; 51(6):440-444.
50. Tamura G, Sato K, Akiyama S, et al. Molecular characterization of undifferentiated-type gastric carcinoma. Lab Invest. 2001; 81(4):593-598.
51. Mateus A R, Seruca R, Machado J C, et al. EGFR regulates RhoA-GTP dependent cell motility in E-cadherin mutant cells. Hum Mol Genet. 2007; 16(13):1639-1647.
52. Deplazes J, Fuchs M, Rauser S, et al. Racl and rho contribute to the migratory and invasive phenotype associated with somatic E-cadherin mutation. Hum Mol Genet. 2009; 18(19):3632-3644.
53. Xu X T, Song Q B, Yao Y, Ruan P, Tao Z Z. Inhibition of RhoA/ROCK signaling pathway promotes the apoptosis of gastric cancer cells. Hepatogastroenterology. 2012; 59(120):2523-2526.
54. Yuan C X, Zhou Z W, Yang Y X, et al. Danusertib, a potent pan-aurora kinase and ABL kinase inhibitor, induces cell cycle arrest and programmed cell death and inhibits epithelial to mesenchymal transition involving the PI3K/akt/mTOR-mediated signaling pathway in human gastric cancer AGS and NCI-N78 cells. Drug Des Devel Ther. 2015; 9:1293-1318.
55. Gamboa-Dominguez A, Dominguez-Fonseca C, Chavarri-Guerra Y, et al. E-cadherin expression in sporadic gastric cancer from mexico: Exon 8 and 9 deletions are infrequent events associated with poor survival. Hum Pathol. 2005; 36(1):29-35.
56. Carpenter P M, Al-Kuran R A, Theuer C P. Paranuaclear E-cadherin in gastric adenocarcinoma. Am J Clin Pathol. 2002; 118(6):887-894.
57. Handschuh G, Candidus S, Luber B, et al. Tumour-associated E-cadherin mutations alter cellular morphology, decrease cellular adhesion and increase cellular motility. Oncogene. 1999; 18(30):4301-4312.
58. Ascano J J, Frierson H, Jr, Moskaluk C A, et al. Inactivation of the E-cadherin gene in sporadic diffuse-type gastric cancer. Mod Pathol. 2001; 14(10):942-949.
59. Gabbert H E, Muller W, Schneiders A, Meier S, Hommel G. The relationship of p53 expression to the prognosis of 418 patients with gastric carcinoma. Cancer. 1995; 76(5):720-726.
60. Kim J P, Oh S T, Hwang T S, Chi J G. The prognostic significance of c-erbB-2 and p53 protein expressions in gastric carcinoma—a multivariate analysis of prognostic factors. J Korean Med Sci. 1994; 9(3):248-253.
61. Monig S P, Eidt S, Zirbes T K, Stippel D, Baldus S E, Pichlmaier H. P53 expression in gastric cancer: Clinicopathological correlation and prognostic significance. Dig Dis Sci. 1997; 42(12):2463-2467.
62. Motojima K, Furui J, Kohara N, Ito T, Kanematsu T. Expression of p53 protein in gastric carcinomas is not independently prognostic. Surgery. 1994; 116(5):890-895.
63. Sasaki I, Yao T, Nawata H, Tsuneyoshi M. Minute gastric carcinoma of differentiated type with special reference to the significance of intestinal metaplasia, proliferative zone, and p53 protein during tumor development. Cancer. 1999; 85(8):1719-1729.
64. Bang Y J, Van Cutsem E, Feyereislova A, et al. Trastuzumab in combination with chemotherapy versus chemotherapy alone for treatment of HER2-positive advanced gastric or gastro-oesophageal junction cancer (ToGA): A phase 3, open-label, randomised controlled trial. Lancet. 2010; 376(9742):687-697.
65. Cristescu R, Lee J, Nebozhyn M. Molecular analysis of gastric cancer identifies subtypes associated with distinct clinical outcomes. Nat Med. 2015; 21(5):449-56.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method comprising:
providing a sample comprising tissue comprising tumor cells from a gastric tumor; and
determining in tumor cells in the sample:
(i) a level of Epstein Barr Virus in the tumor cells;
(ii) DNA mismatch repair protein expression in the tumor cells;
(iii) E-cadherin protein expression in the tumor cells;
(iv) p53 protein expression in the tumor cells; and
(v) Mucin protein expression in the tumor cells.

2. The method of claim 1, further comprising:
categorizing a tumor with EBV, preferably nuclear EBV, above a reference level, and optionally with prominent lymphoid infiltrate, in group 1;
categorizing a tumor with a nuclear DNA mismatch repair protein expression below a reference level in group 2;
categorizing a tumor with E-cadherin expression below a reference level, or only cytoplasmic E-cadherin expression, in group 3;
categorizing a tumor with normal p53 expression and cytoplasmic mucin expression above a reference level in group 4; and
categorizing a tumor with aberrant levels of p53 expression and normal levels of mucin expression in group 5.

3. The method of claim 1, wherein the level of Epstein Barr Virus is determined using EBER in situ hybridization (EBER-ISH), and/or the DNA mismatch repair protein expression; E-cadherin expression; p53 expression; and Mucin expression is detected using immunohistochemistry in intact tumor samples.

4. The method of claim 1, wherein the mismatch repair proteins comprise one, two, three, or all four of MLH1, PMS2, MSH2, or MSH6.

5. The method of claim 4, wherein the mismatch repair proteins comprise MLH1 and PMS2.

6. The method of claim 1, wherein the Mucin is one or more of MUC2, CDX2, CD10, MUCSAC, or MUC6.

7. The method of claim 6, wherein the Mucin is MUC6.

8. The method of claim 2, wherein aberrant p53 expression is an absence of expression or strong diffuse expression, and normal p53 expression is weak, patchy expression.

9. The method of claim 2, further comprising selecting, and optionally administering,
a treatment comprising a therapeutically effective amount of an immunotherapy, preferably a checkpoint inhibitor, to a subject having a tumor in group 1;
a treatment comprising a therapeutically effective amount of an immunotherapy, topoisomerase-I inhibitors, or platinum-based chemotherapy, optionally in combination with poly (ADP-ribose) polymerase (PARP) inhibitors, to a subject having a tumor in group 2;
a treatment comprising a therapeutically effective amount of an inhibitor of mammalian target of rapamycin (mTOR) to a subject having a tumor in group 3;
a treatment comprising a therapeutically effective amount of an antimetabolite to a subject having a tumor in group 4; or
a treatment comprising a therapeutically effective amount of an inhibitor of polo-like kinase 1 (PLK1) and/or an inhibitor of Aurora Kinase A to a subject having a tumor in group 5.

10. The method of claim 9, wherein the checkpoint inhibitor is an anti-PD1 or anti-PDL1 antibody.

11. The method of claim 9, wherein the mTOR inhibitor is rapamycin, ridaforolimus, temsirolimus, everolimus, sirolimus, dactolisib, AZD8055, CZ415, CC-223, voxtalisib, PI-103, KU-0063794, torkinib, ridaforolimus, Voxtalisib (SAR245409, XL765) analogue, omipalisib, OSI-027, PF-04691502, Apitolisib (GDC-0980, RG7422), GSK1059615, Gedatolisib (PF-05212384, PKI-587), WYE-354, Vistusertib (AZD2014), Torin 2, WYE-125132 (WYE-132), BGT226 (NVP-BGT226), Palomid 529 (P529), PP121, WYE-687, CH5132799, WAY-600. ETP-46464. GDC-0349, XL388, zotarolimus, MHY1485 and Chrysophanic Acid.

12. The method of claim 9, wherein the antimetabolite is 5-Fluorouracil (5-FU), gemcitabine, fluorouracil, cytarabine, cladribine, methotrexate, pemetrexed, capecitabine, hydroxyurea, fludarabine, pralatrexate, nelarabine, cloafarabine, decitabine, fluxuridine, and thioguanine.

13. The method of claim 9, wherein the antimetabolite is a pyrimidine analog, preferably 5-Fluorouracil (5-FU), gemcitabine, fluorouracil, or cytarabine.

14. The method of claim 9, wherein the inhibitor of polo-like kinase 1 (PLK1) is volasertib, BI 2536, GSK461364, NMS-P937 (NMS1286937), Ro3280, MLN0905, SBE 13 HCl, HMN-214, HMN-176, or rigosertib.

15. The method of claim 9, wherein the inhibitor of inhibitor of Aurora Kinase A is MK-5108 (VX-689), MK-8745, Alisertib (MLN8237), Aurora A Inhibitor I, MLN8054, ENMD-2076 L-(+)-Tartaric acid, ENMD-2076, KW-2449, VX-680 (Tozasertib, MK-0457), PF-03814735, AT9283, AMG-900, CYC116, SNS-314 Mesylate, JNJ-7706621, Reversine, Danusertib (PHA-739358), CCT137690, TAK-901, PHA-680632, CCT129202, ZM 447439, GSK1070916, Barasertib (AZD1152-HQPA), alisertib (MLN8327), or Hesperadin.

16. A method of treating a gastric tumor in a subject, the method comprising:
providing a sample comprising tissue comprising tumor cells from the gastric tumor; and
determining in tumor cells in the sample:
(i) a level of Epstein Barr Virus (EBV) in the tumor cells, preferably a nuclear level of EBV;
(ii) DNA mismatch repair protein expression in the tumor cells;
(iii) E-cadherin protein expression in the tumor cells;
(iv) p53 protein expression in the tumor cells; and
(v) Mucin protein expression in the tumor cells; and
categorizing a tumor with EBV, preferably nuclear EBV, above a reference level, and optionally with prominent lymphoid infiltrate, in group 1;
categorizing a tumor with a nuclear DNA mismatch repair protein expression below a reference level in group 2;
categorizing a tumor with E-cadherin expression below a reference level, or only cytoplasmic E-cadherin expression, in group 3;
categorizing a tumor with normal p53 expression and cytoplasmic mucin expression above a reference level in group 4; and
categorizing a tumor with aberrant levels of p53 expression and normal levels of mucin expression in group 5, and
administering a treatment comprising a therapeutically effective amount of an immunotherapy, preferably a checkpoint inhibitor, to a subject having a tumor in group 1;
administering a treatment comprising a therapeutically effective amount of an immunotherapy, topoisomerase-I inhibitors, or platinum-based chemotherapy, optionally in combination with poly (ADP-ribose) polymerase (PARP) inhibitors, to a subject having a tumor in group 2;
administering a treatment comprising a therapeutically effective amount of an inhibitor of mammalian target of rapamycin (mTOR) to a subject having a tumor in group 3;
administering a treatment comprising a therapeutically effective amount of an antimetabolite to a subject having a tumor in group 4; or
administering a treatment comprising a therapeutically effective amount of an inhibitor of polo-like kinase 1 (PLK1) and/or an inhibitor of Aurora Kinase A to a subject having a tumor in group 5.

17. The method of claim 16, wherein:
the checkpoint inhibitor is an anti-PD1 or anti-PDL1 antibody;
the mTOR inhibitor is rapamycin, ridaforolimus, temsirolimus, everolimus, sirolimus, dactolisib, AZD8055, CZ415, CC-223, voxtalisib, PI-103, KU-0063794, torkinib, ridaforolimus, Voxtalisib (SAR245409, XL765) analogue, omipalisib, OSI-027, PF-04691502, Apitolisib (GDC-0980, RG7422), GSK1059615, Gedatolisib (PF-05212384, PKI-587), WYE-354, Vistusertib (AZD2014), Torin 2, WYE-125132 (WYE-132), BGT226 (NVP-BGT226), Palomid 529 (P529), PP121, WYE-687, CH5132799, WAY-600. ETP-46464. GDC-0349, XL388, zotarolimus, MHY1485 and Chrysophanic Acid;
the antimetabolite is 5-Fluorouracil (5-FU), gemcitabine, fluorouracil, cytarabine, cladribine, methotrexate, pemetrexed, capecitabine, hydroxyurea, fludarabine, pralatrexate, nelarabine, cloafarabine, decitabine, fluxuridine, and thioguanine;
the inhibitor of polo-like kinase 1 (PLK1) is volasertib, BI 2536, GSK461364, NMS-P937 (NMS1286937), Ro3280, MLN0905, SBE 13 HCl, HMN-214, HMN-176, or rigosertib;
and/or
the inhibitor of inhibitor of Aurora Kinase A is MK-5108 (VX-689), MK-8745, Alisertib (MLN8237), Aurora A Inhibitor I, MLN8054, ENMD-2076 L-(+)-Tartaric acid, ENMD-2076, KW-2449, VX-680 (Tozasertib, MK-0457), PF-03814735, AT9283, AMG-900, CYC116, SNS-314 Mesylate, JNJ-7706621, Reversine, Danusertib (PHA-739358), CCT137690, TAK-901, PHA-680632, CCT129202, ZM 447439, GSK1070916, Barasertib (AZD1152-HQPA), alisertib (MLN8327), or Hesperadin.

18. The method of claim 17, wherein the antimetabolite is a pyrimidine analog, preferably 5-Fluorouracil (5-FU), gemcitabine, fluorouracil, or cytarabine.
